(19) United States
(12) Patent Application Publication
(10) Pub. No.: US 2026/0335480 A1
(43) Pub. Date: Oct. 29, 2026

(54) DEODORIZING SOLUTION FOR NEUTRALIZING ODORS AND METHOD THEREFOR

(71) Applicant: Pamela Romero, Eagle, ID (US)

(72) Inventor: Pamela Romero, Eagle, ID (US)

(21) Appl. No.: 19/186,790

(22) Filed: Apr. 23, 2025

(51) Int. Cl. A61L 2/18; A61L 101/32; A61L 101/34; A61L 101/36; A61L 101/44; A61L 103/50; D06M 13/00; D06M 13/03; D06M 13/13; D06M 13/144; D06M 13/224; D06M 13/228

(52) U.S. Cl. CPC A61L 2/18; D06M 13/005; D06M 13/03; D06M 13/13; D06M 13/144; D06M 13/2246; D06M 13/228; A61L 2101/32; A61L 2101/34; A61L 2101/36; A61L 2101/44; A61L 2103/50

(57) ABSTRACT

A deodorizing solution for neutralizing odors includes 91%-99% isopropyl alcohol, coumarin, benzyl benzoate, limonene, hexyl cinnamal, and water. The deodorizing agent can be used to treat various odors, including, but not limited to, pet odors, smoke odors, and bacteria-produced odors, such as sweat odor or body odor.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to cleaning solutions and more particularly pertains to a new deodorizing solution for neutralizing odors, such as pet urine odors, smoke odors, and body odors.

(2) Description of Related Art including information disclosed under 37 CFR 1.97 and 1.98.

The prior art relates to cleaning solutions. The prior art, as best understood, does not disclose a deodorizing solution for neutralizing odors that includes 91%-99% isopropyl alcohol, coumarin, benzyl benzoate, limonene, hexyl cinnamal, and water.

### (h) BRIEF SUMMARY OF THE INVENTION

An embodiment of the disclosure meets the needs presented above in a deodorizing solution generally comprising 91%-99% isopropyl alcohol, coumarin, benzyl benzoate, limonene, hexyl cinnamal, and water.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a deodorizing solution according to an embodiment of the disclosure.

FIG. 2 is a side view of an embodiment of the disclosure in use.

FIG. 3 is a block diagram of components of an embodiment of the disclosure.

### (J) DETAILED DESCRIPTION OF THE INVENTION

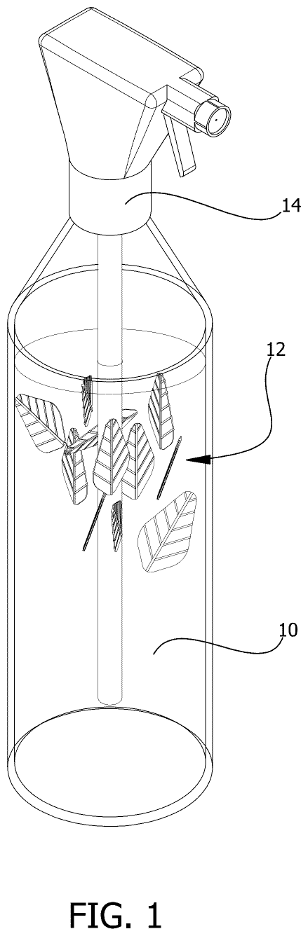

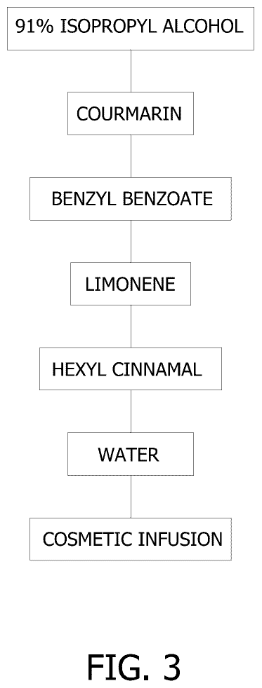

With reference now to the drawings, and in particular to FIGS. 1 through 3 thereof, a new deodorizing solution embodying the principles and concepts of an embodiment of the disclosure and generally designated by the reference numeral 10 will be described.

As best illustrated in FIGS. 1 through 3, the deodorizing solution 10 for neutralizing odors generally comprises 91%-99% isopropyl alcohol, coumarin, benzyl benzoate, limonene, hexyl cinnamal, and water.

The isopropyl alcohol, which can be 91% isopropyl alcohol or greater, is in an amount and/or concentration sufficient to act as a disinfectant, solvent, and quick-drying agent to enhance odor elimination and microbial control. The coumarin is in an amount and/or concentration sufficient to provide a warm, sweet fragrance and aid in masking residual odors. The benzyl benzoate is in an amount and/or concentration sufficient to act as a fragrance fixative and antimicrobial agent to prolong the effectiveness of the scent. The limonene is a naturally derived citrus-scented compound, which is in an amount and/or concentration sufficient to contribute to odor neutralization and enhance degreasing properties. The hexyl cinnamal is a floral fragrance component, which is in an amount and/or concentration sufficient to enhance the overall scent profile of the deodorizing solution 10. The water is in a relatively small amount by volume but is an amount and/or concentration sufficient to balance the formulation and control volatility.

In the exemplary embodiment in FIGS. 1 through 3, the deodorizing solution 10 further includes potpourri 12. The potpourri 12, as is well known, is a mixture of dried, naturally fragrant plant materials used to produce a pleasant scent. Some examples of potpourri 12 ingredients include dried flowers and petals, such as roses, lavender, chamomile, and hibiscus; dried leaves and herbs, such as eucalyptus, mint, rosemary, or sage; dried fruits and peels, such as orange, lemon, and apple slices; spices, such as cinnamon sticks, cloves, star anise, and nutmeg; and wood and bark, such as cedarwood chips or sandalwood shavings. In the exemplary embodiment in FIG. 1, potpourri 12 in the form of dried leaves or petals is shown floating in the deodorizing solution 10 to enhance the visual aesthetic of the deodorizing solution 10 and possibly provide fragrance properties, though other potpourri 12 materials could be used in another possible embodiment. In another possible embodiment, the potpourri 12 could be omitted.

The different components or ingredients of the deodorizing solution 10 can be present in different concentrations as would be suitable to produce different effects. In one possible embodiment, the isopropyl alcohol has a concentration in the range of 80% to 95% by volume, which can optimize disinfecting and drying properties of the deodorizing solution 10. In another possible embodiment, the coumarin has a concentration in the range of 0.01% to 0.5% by volume, which can promote safe usage of the deodorizing solution 10 in household environments. In another possible embodiment, the benzyl benzoate has a concentration in the range of 0.1% to 2% by volume, which can enhance longevity of the fragrance effect of the deodorizing solution 10. In another possible embodiment, the limonene has a concentration in the range of 0.1% to 1.5% by volume, which can aid in odor removal and provide a fresh citrus scent of the deodorizing solution 10. In another possible embodiment, the hexyl cinnamal has a concentration in the range of 0.05% to 0.3% by volume, which can provide a minimized floral fragrance of the deodorizing solution 10. The preceding ranges include all values in tenths of a percent and hundredths of a percent. It should be understood that the concentrations can be adjusted as desired to increase or decrease a desired effect. For example, the concentration of hexyl cinnamal could be increased to increase the floral fragrance produced by the hexyl cinnamal, or the isopropyl alcohol could be increased for greater disinfecting and drying effects.

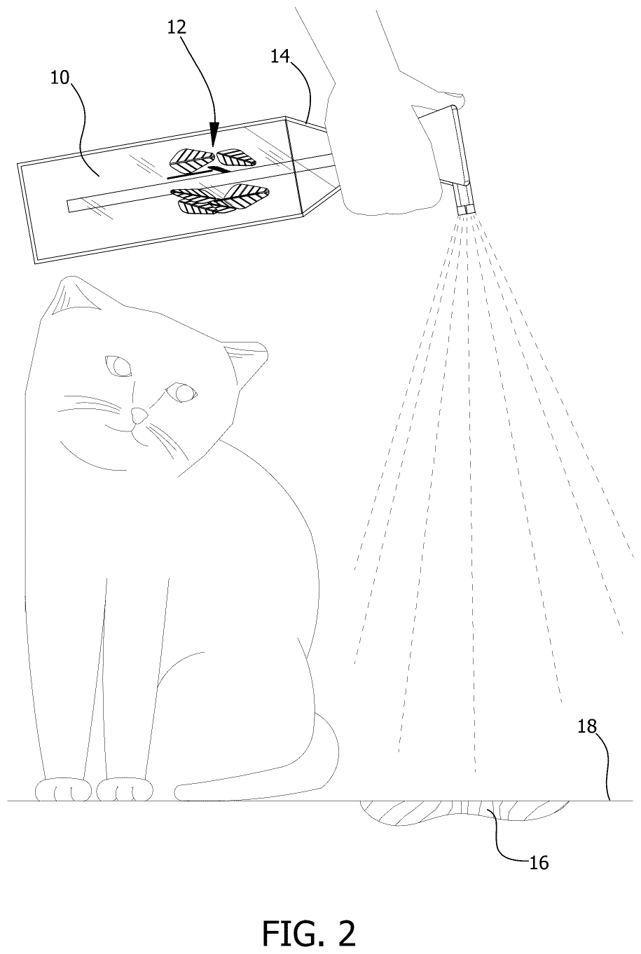

In the exemplary embodiment in FIG. 2, the deodorizing solution 10 is used to treat a pet urine stain and the resulting odor. The deodorizing solution 10 is in a spray bottle 14. A user applies the deodorizing solution 10, such as by spraying, onto a surface, such as carpeting 18, that has a pet urine stain 16. The user then allows the deodorizing solution 10 to dwell on the surface for a period sufficient for the deodorizing solution 10 to neutralize odors. The user then either wipes the surface dry or allows the surface to air dry. In another possible embodiment, the deodorizing solution 10 can be used to treat a variety of odors, including, but not limited to, pet odors, smoke odors, and bacteria-produced odors, such as sweat odor or body odor.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A deodorizing solution for neutralizing odors comprising:
91%-99% isopropyl alcohol;
coumarin;
benzyl benzoate;
limonene;
hexyl cinnamal; and
water.

2. The deodorizing solution of claim 1, further comprising potpourri.

3. The deodorizing solution of claim 1, wherein the isopropyl alcohol has a concentration in the range of 80% to 95% by volume.

4. The deodorizing solution of claim 1, wherein the coumarin has a concentration in the range of 0.01% to 0.5% by volume.

5. The deodorizing solution of claim 1, wherein the benzyl benzoate has a concentration in the range of 0.1% to 2% by volume.

6. The deodorizing solution of claim 1, wherein the limonene has a concentration in the range of 0.1% to 1.5% by volume.

7. The deodorizing solution of claim 1, wherein the hexyl cinnamal has a concentration in the range of 0.05% to 0.3% by volume.

8. The deodorizing solution of claim 3, wherein the coumarin has a concentration in the range of 0.01% to 0.5% by volume.

10. The deodorizing solution of claim 9, wherein the benzyl benzoate has a concentration in the range of 0.1% to 2% by volume.

11. The deodorizing solution of claim 10, wherein the limonene has a concentration in the range of 0.1% to 1.5% by volume.

12. The deodorizing solution of claim 11, wherein the hexyl cinnamal has a concentration in the range of 0.05% to 0.3% by volume.

13. The deodorizing solution of claim 2, wherein the isopropyl alcohol has a concentration in the range of 80% to 95% by volume.

14. The deodorizing solution of claim 13, wherein the coumarin has a concentration in the range of 0.01% to 0.5% by volume.

15. The deodorizing solution of claim 14, wherein the benzyl benzoate has a concentration in the range of 0.1% to 2% by volume.

16. The deodorizing solution of claim 15, wherein the limonene has a concentration in the range of 0.1% to 1.5% by volume.

17. The deodorizing solution of claim 16, wherein the hexyl cinnamal has a concentration in the range of 0.05% to 0.3% by volume.

18. A method of treating pet urine stains and neutralizing odors, the method comprising the steps of:
applying to a surface a deodorizing solution comprising:
91%-99% isopropyl alcohol;
coumarin;
benzyl benzoate;
limonene;
hexyl cinnamal; and
water;
allowing the deodorizing solution to dwell on the surface for a period sufficient for the deodorizing solution to neutralize odors; and
wiping the surface dry or allowing the surface to air dry.

19. A method of neutralizing odors including pet odors, smoke odors, and bacteria-produced odors, the method comprising the steps of:
applying to a surface a deodorizing solution comprising:
91%-99% isopropyl alcohol;
coumarin;
benzyl benzoate;
limonene;
hexyl cinnamal; and
water;
allowing the composition to dwell on the surface for a period sufficient for odor-neutralizing action; and
wiping the surface or allowing the surface to air dry.
